(19) United States
(12) Patent Application Publication
(10) Pub. No.: US 2023/0390106 A1
(43) Pub. Date: Dec. 7, 2023

(54) Smart Heating Pad System

(71) Applicant: I-SYST inc., Brossard (CA)

(72) Inventor: Nguyen Hoan Hoang, Brossard (CA)

(73) Assignee: I-SYST inc., Brossard (CA)

(21) Appl. No.: 18/328,425

(22) Filed: Jun. 2, 2023

Related U.S. Application Data
(60) Provisional application No. 63/348,272, filed on Jun. 2, 2022.

(51) Int. Cl. A61F 7/02; G16H 40/67

(52) U.S. Cl. CPC A61F 7/02; G16H 40/67; A61F 2007/0093

(57) ABSTRACT

A smart heating pad system is provided. The smart heating pad system includes a plurality of individual heating pads that are secured within a garment. Each heating pad includes a PCB having a USB connector, a wireless transceiver, and a microprocessor. Each heating pad includes dedicated batteries, or the heating pads are all operably connected to a single battery power supply. The microprocessor is operably connected to the temperature sensor, allowing temperature data to be transmitted to an external control device, such as a smartphone. The external control device also wirelessly adjust the temperature of the pads. The system provides the ability to independently monitor and adjust temperatures along different areas of the body depending on the type of garment in which the heating pads are secured.

### CROSS REFERENCE TO RELATED APPLICATIONS

This application claims the benefit of U.S. Provisional Application No. 63/348,272 filed on Jun. 2, 2022. The above identified patent application is herein incorporated by reference in its entirety to provide continuity of disclosure.

### BACKGROUND OF THE INVENTION

The present invention relates to a heating pad. More specifically, the present invention relates to a smart heating pad system.

Many individuals desire additional heating options when clothing or garments are unable to keep them warm, due to the material, the environment, or other factors. Heated garments have been developed, but they have many drawbacks. For example, most heated garments include a single heating wire that may not be adjusted and may only be turned on and off.

This may result in the garment either becoming too hot or not hot enough in some cases. Even in garments with multiple heating elements, these may typically only be controlled as a whole, which does not allow for fine tuning or adjustment. Further, most heated garments also fail to provide the user with temperature information, which may be important for monitoring for health purposes and for determining an ideal personal temperature for the heaters.

In view of the above concerns, it is desirable to provide a heating pad system that may be wirelessly monitored and allow for control of individual heating pads within a network of heating pads.

In light of the devices disclosed in the known art, it is submitted that the present invention substantially diverges in design elements from the known art and consequently it is clear that there is a need in the art for an improvement to existing smart heating pad systems and similar devices. In this regard the present invention substantially fulfills these needs.

### SUMMARY OF THE INVENTION

In view of the foregoing disadvantages inherent in the known types of smart heating pad systems now present in the prior art, the present invention provides a smart heating pad system wherein the same may be utilized for providing convenience for the user when using a smart heating pad system.

The present system comprises a smart heating pad system having a heating pad having a plurality of heating elements attached to a base, and a printed circuit board attached to one edge of the base, wherein the printed circuit board includes an attached dedicated battery, a thermal sensor, a wireless transceiver, a microprocessor, a microcontroller, a wireless communication technology, and motion sensors, wherein the heating pad temperature is monitored and adjusted via an external device receiving and sending wireless information, and wherein the printed circuit board includes charging ports for recharging the attached dedicated battery.

The present system comprises a smart heating pad system having a heating pad having a plurality of heating elements disposed on a top surface of the heating pad, a plurality of connecting pins connecting a PCB to the heating pad, and a smart battery having the microcontroller, the wireless transceiver, a cellular and/or satellite wireless gateway, the temperature sensor, a rechargeable battery, a 9-axis Inertial measurement unit with a gyroscope, an accelerometer, a magnetometer, a GPS, and a plurality of power and communication lines.

### BRIEF DESCRIPTION OF THE DRAWINGS

The invention itself and manner in which it may be made and used may be better understood after a review of the following description, taken in connection with the accompanying figures.

FIG. 1 shows a top view of a heating pad from one embodiment of the smart heating pad system.

FIG. 2 shows a bottom view of a heating pad from one embodiment of the smart heating pad system.

FIG. 3 shows a top view of an external control device adjusting the temperature in one embodiment of the smart heating pad system.

FIG. 4 shows a perspective view of an alternate embodiment of the smart heating pad system.

FIG. 5 shows a top view of an external control device adjusting the temperature in an alternate embodiment of the smart heating pad system.

FIG. 6 shows a top view of a pair of heating pads from one embodiment of the smart heating pad system.

FIG. 7 shows a top view of a heating pad and a PCB from one embodiment of the smart heating pad system.

FIG. 8 shows an electrical diagram of a smart battery from one embodiment of the smart heating pad system.

Reference is made herein to the attached drawings. Like reference numerals are used throughout the drawings to depict like or similar elements of the smart heating pad system. The figures are intended for representative purposes only and should not be considered to be limiting in any respect.

### DETAILED DESCRIPTION OF THE INVENTION

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

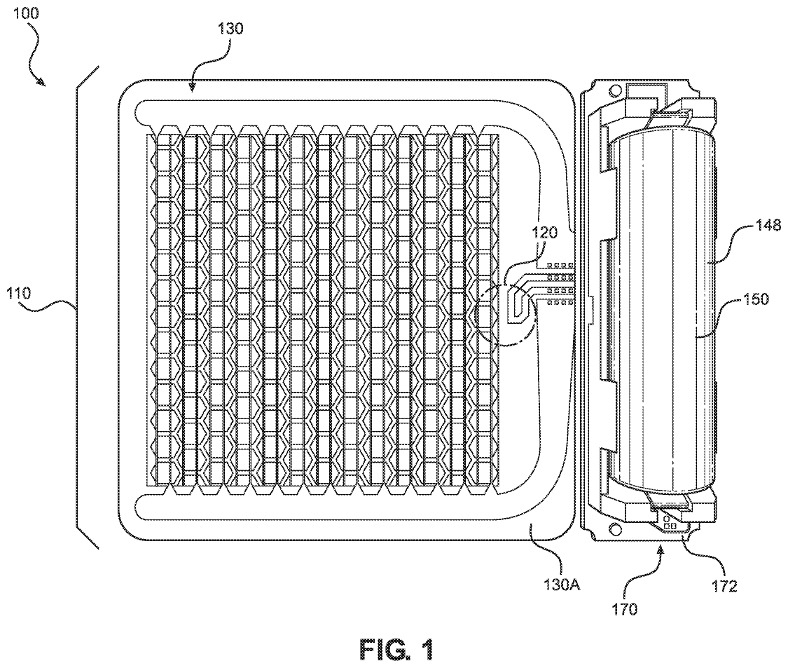

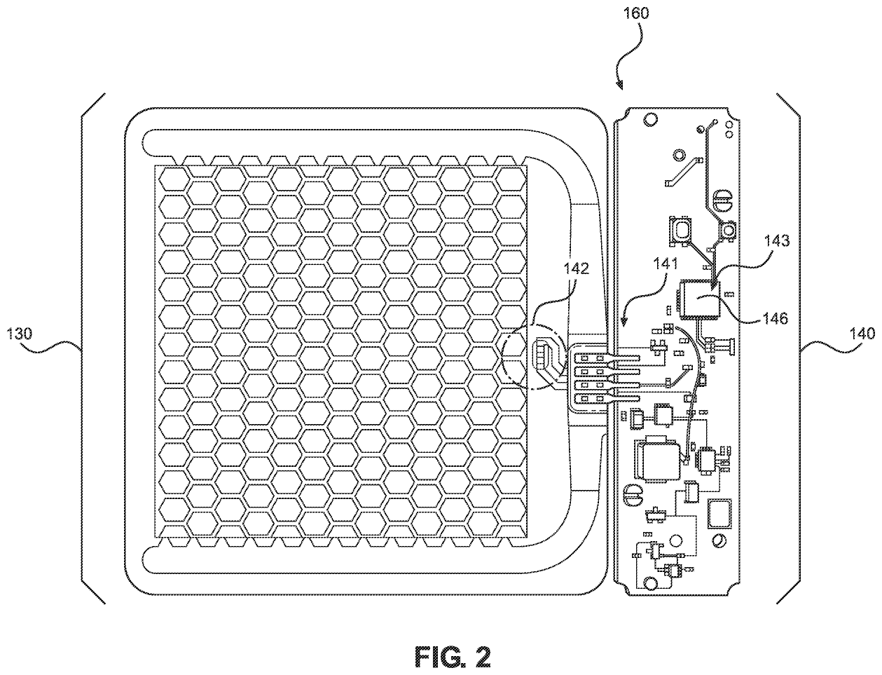

Referring now to FIGS. 1 and 2, there is shown a heating pad 110 from one embodiment of the smart heating pad system 100.

In the shown embodiment, the heating pad 110 may include a plurality of heating elements 120 attached to a base 130, with a printed circuit board or PCB 140 attached to one edge 130A of the base 130. The PCB 140 may include an attached dedicated battery 150 that powers electrical components 160 of the heating pad 110. The PCB 140 may include charging ports 170, such as USB ports 172 or the like, for recharging the attached dedicated battery 150. The PCB 140 may include a thermal sensor 142 and a microprocessor 146. The microprocessor 146 may be programmed to allow the thermal sensor 142 to measure current temperature and wirelessly transmit temperature data to an external device 180 via a wireless transceiver 143. The heating pad 110 may be covered by an insulating fabric or otherwise thermal regulating fabric such as Merino wool or graphene fabric, for example. Further, the PCB 140 may be enclosed in a housing 148 for protection, which may include an opening for the USB ports 172 or other ports for recharging the attached dedicated battery 150.

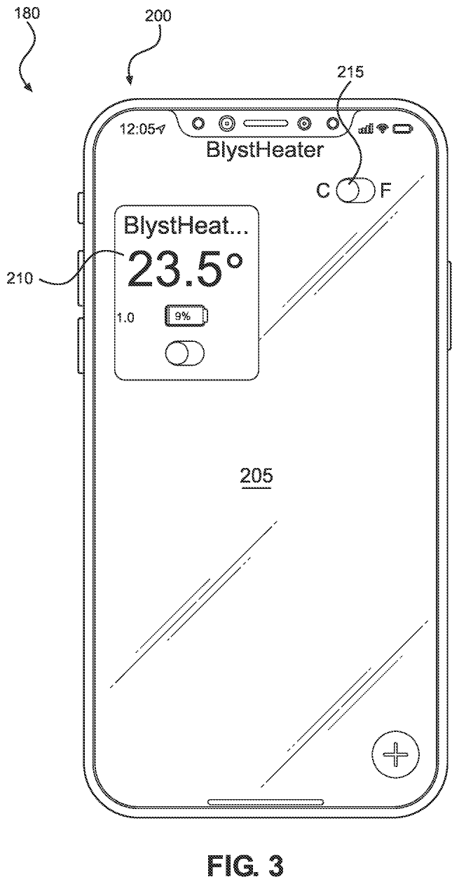

Referring now to FIG. 3, there is shown a top view of an external device 180 adjusting the temperature in one embodiment of the smart heating pad system 100. FIG. 3 shows a smart phone interface 205 showing a temperature of 23.5° C. with a battery charged at 9 percent and a toggle switch 215 between Celsius (C°) and Fahrenheit (F°).

The heating pad 110 temperature may be monitored and adjusted via the external device 180 such as a Smartphone 200 or any other computing device capable of receiving and sending wireless information. A non-transitory storage media 210 may allow the external device 180 to remotely control the temperature levels of the heating pad 110. The non-transitory storage media 210 may display the temperature data and other sensory data received from the heating pad 110.

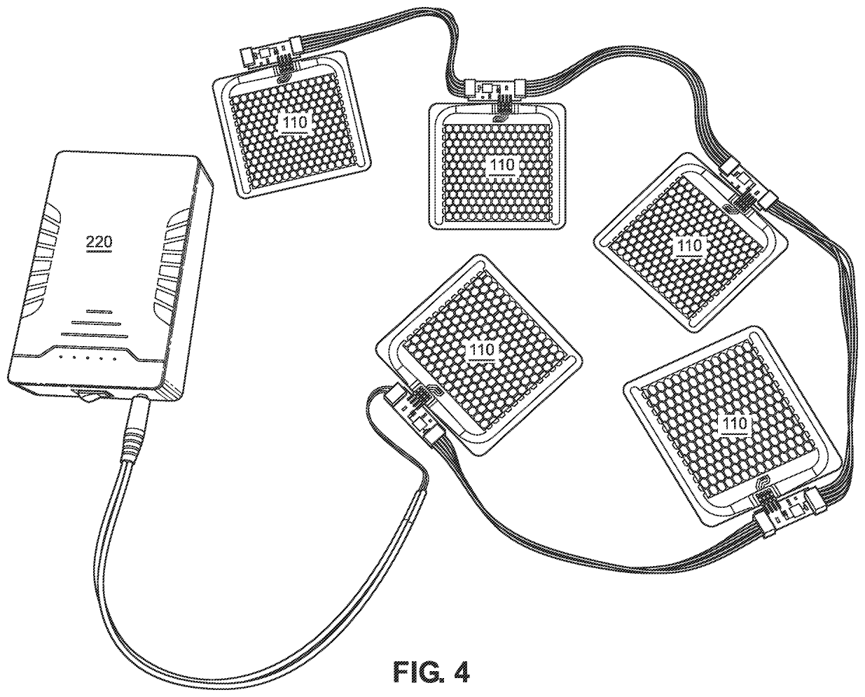

Referring now to FIG. 4, there is shown a perspective view of an alternate embodiment of the smart heating pad system 100. FIG. 4 shows 5 heating pads 110 associated with the smart heating pad system 100.

In the shown embodiment, the heating pads 110 are electrically connected to one another in a mesh network topology and are powered by a smart battery pack 220. The smart battery pack 220 may include multiple batteries in the form of canisters or pouches (not shown) connected to the PCB 140 comprising its own microcontroller 141, wireless communication technology 142, multi-axis intertial measurement unit (IMU) 145, a GPS system 227, and a cellular and/or satellite communications module(s) 221. This smart battery pack 220 may be responsible for controlling the smart heating pad system 100 as well as sending power to the heating pad 110 connected to it. The heating pad 110 within the network of heating pads 110, independent temperature sensors 149, and data from them is communicated to the smart battery pack 220, which is then transmitted to the external device 180 via the wireless transceiver 142 of the smart battery pack 220.

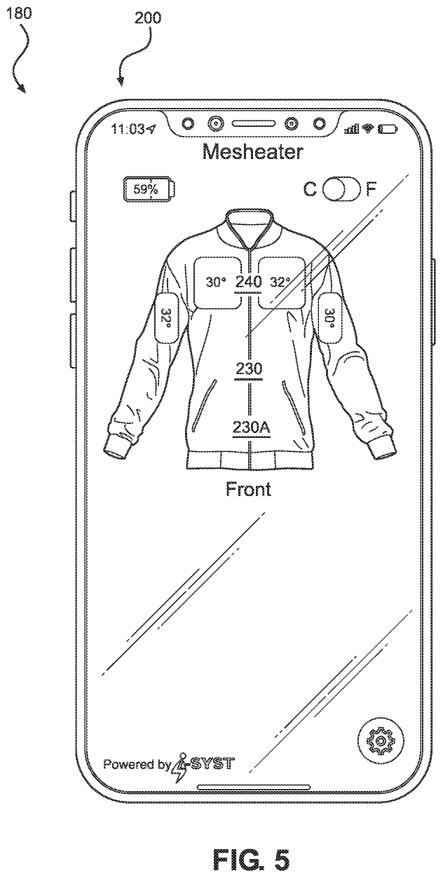

Referring now to FIG. 5, there is shown a top view of the external device 180 adjusting the temperature in an alternate embodiment of the smart heating pad system 100.

In the shown embodiment, the heating pads 110 are installed at various locations within a garment 230. The garment 230 as shown in FIG. 5 is a jacket 230A, but the smart heating pad system 100 be utilized in the garment 230 or other devices, such as a blanket, as one example. The external device 180 may be configured to display a visual map 240 of the heating pads 110 as they are located in the garment 230, while also giving the user the ability to monitor and control the temperatures of each of the heating pads 110. In this way, the smart heating pad system 100 allows users to more precisely monitor and adjust heat levels to suit their personal preferences.

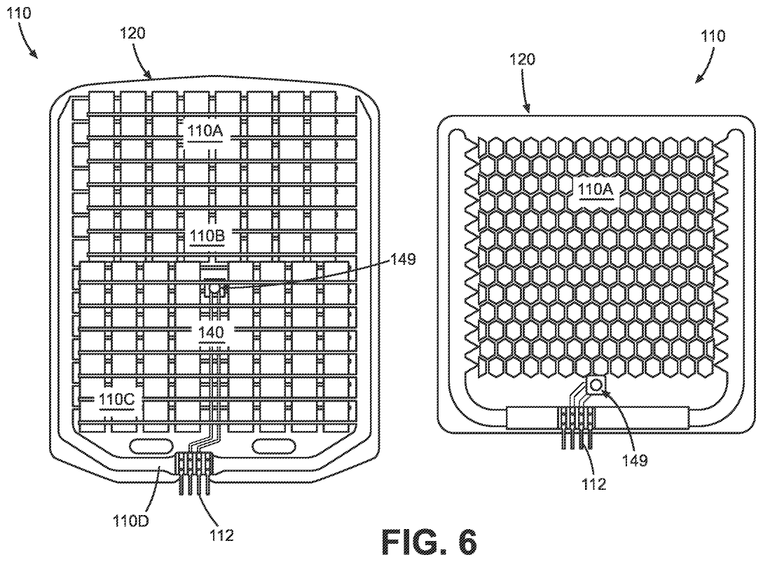

Referring now to FIG. 6, there is shown a top view of a pair of heating pads 110. The heating pads 110 can be of any size and shape.

The pair of heating pads 110 may each include the heating elements 120 disposed on a top surface 110A of the heating pad 110. The heating pad 110 may include a temperature sensor 149 disposed on a middle portion 110B of the heating pad 110 or disposed on an edge 110C of the heating pad 110. Each of the pair of heating pads 110 may include a plurality of connecting pins 112 that may connect the PCB 140 to the heating pad 110. The connecting pins 112 may be disposed on an outer edge 110D of the heating pad 110 that may connect the PCB 140 to the heating pad 110.

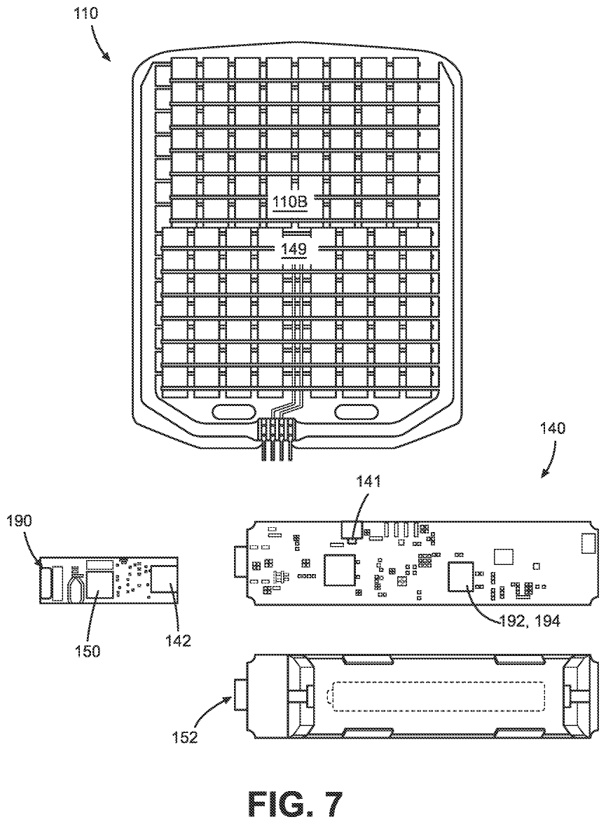

Referring now to FIG. 7, there is shown a top view of the heating pad 110 and the PCB 140.

The heating pad 110 may include the temperature sensor 149 disposed on a middle portion 110B of the heating pad 110.

The PCB 140 may include an attached dedicated battery (FIG. 2, 150) disposed on the PCB 140. The PCB 140 may include a battery holder 152 that may contain the attached dedicated battery (FIG. 2, 150) disposed on the PCB 140.

The PCB 140 may be a networked PCB 190 that is in communication with a network 192 such as the Internet 194 or the like to operate the heating pad 110 in a client server environment and/or an Internet of Things environment.

The networked PCB 190 may include a microprocessor (FIG. 2, 146) and a wireless transceiver (FIG. 4, 143) to enable operating the heating pad 110 in a client server environment and/or an Internet of Things environment.

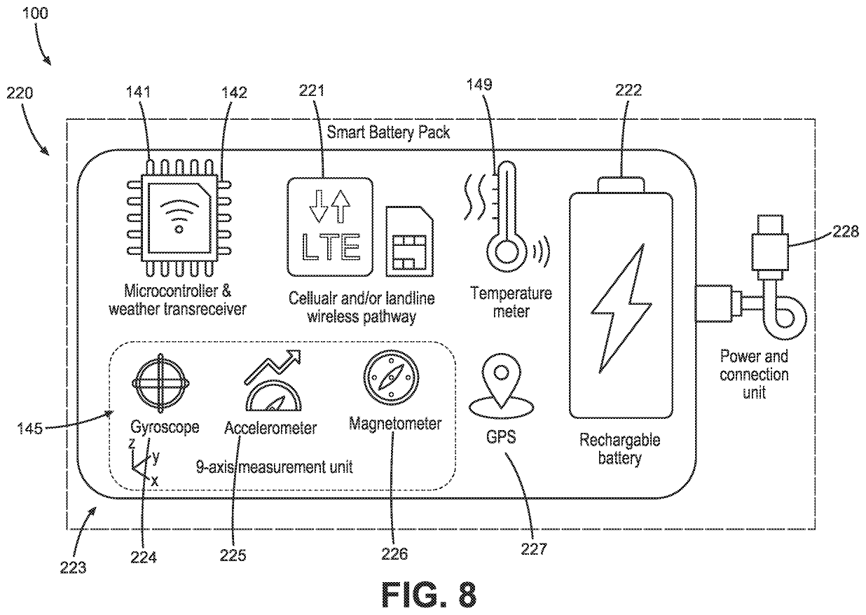

FIG. 8 shows an electrical diagram of a smart battery (FIG. 4, 220) from one embodiment of the smart heating pad system 100.

The smart battery 220 may include a microcontroller 141 and a wireless transceiver 142, a cellular and/or satellite wireless gateway 221, a temperature sensor 149, a rechargeable battery 222, a 9-axis Inertial measurement unit 223, a gyroscope 224, an accelerometer 225, a magnetometer 226, a Global Positioning System or GPS 227, and a plurality of power and communication lines 228.

The microcontroller 141 and the wireless transceiver 142, the cellular and/or satellite wireless gateway 221, the temperature sensor 149, the rechargeable battery 222, the 9-axis Inertial measurement unit 223, the gyroscope 224, the accelerometer 225, the magnetometer 226, and the GPS 227 may be contained in the battery holder 152. The power and communication lines 228 may extend from the battery holder 152.

It is therefore submitted that the present invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A smart heating pad system, comprising:
a heating pad having a plurality of heating elements attached to a base; and
a printed circuit board attached to one edge of the base;
wherein the printed circuit board includes an attached dedicated battery, a thermal sensor, a wireless transceiver, a microprocessor, a microcontroller, a wireless communication technology, and a plurality of motion sensors;
wherein the heating pad temperature is monitored and adjusted via an external device receiving and sending wireless information; and
wherein the printed circuit board includes a plurality of charging ports for recharging the attached dedicated battery.

2. The smart heating pad system, according to claim 1, wherein the external device is a computing device such as a Smartphone, a smart watch, a tablet, or any other computing device capable of receiving and sending wireless information.

3. The smart heating pad system, according to claim 1, wherein the external device displays a visual map of the heating pads as they are located in the garment, while monitoring and controlling temperatures of each of the heating pads.

4. The smart heating pad system, according to claim 1, wherein the garment is a jacket.

5. The smart heating pad system, according to claim 1, wherein the garment is a blanket, a boot, a shoe, a scarf, a belt, a pair of pants, a wet suit, a dry suit, or a diving vest.

6. The smart heating pad system, according to claim 1, wherein the heating pad is covered by a material selected from the group consisting of an insulating fabric, a thermal regulating fabric, Merino wool, or graphene fabric.

7. The smart heating pad system, according to claim 1, wherein the charging ports are USB ports.

8. The smart heating pad system, according to claim 1, wherein the printed circuit board is enclosed in a housing for protection.

9. The smart heating pad system, according to claim 1, wherein the printed circuit board includes an attached dedicated battery disposed on the PCB.

10. The smart heating pad system, according to claim 8, wherein the housing includes an opening for the ports for recharging the attached dedicated battery.

11. The smart heating pad system, according to claim 1, wherein the microprocessor is programmed to control and maintain the temperature of the heating pad according to the temperature set by the external device.

12. A smart heating pad system, comprising:
a heating pad having a plurality of heating elements disposed on a top surface of the heating pad;
a plurality of connecting pins connecting a PCB to the heating pad; and
a smart battery having the microcontroller, the wireless transceiver, a cellular and/or satellite wireless gateway, the temperature sensor, a rechargeable battery, a 9-axis Inertial measurement unit with a gyroscope, an accelerometer, and a magnetometer, a GPS, and a plurality of power and communication lines.

13. The smart heating pad system, according to claim 11, wherein the heating pad includes a temperature sensor disposed on a middle portion of the heating pad.

14. The smart heating pad system, according to claim 12, wherein the heating pad includes the temperature sensor disposed on an edge of the heating pad.

15. The smart heating pad system, according to claim 11, wherein the connecting pins are disposed on an outer edge of the heating pad that connect the PCB to the heating pad.

16. The smart heating pad system, according to claim 11, wherein the PCB includes an attached dedicated battery disposed on the PCB.

17. The smart heating pad system, according to claim 11, wherein the microprocessor is programmed to control and maintain the temperature of the heating pad according to the temperature set forth by the external device.

18. The smart heating pad system, according to claim 11, wherein the PCB is a networked PCB that is in communication with a network that includes the Internet to operate the heating pad in a client server environment.

19. The smart heating pad system, according to claim 11, wherein the PCB is a networked PCB that is in communication with a network that includes the Internet to operate the heating pad in an Internet of Things environment, wherein the microprocessor is programmed to control and maintain the temperature of the heating pad according to the temperature set forth by the external device.

20. The smart heating pad system, according to claim 17, wherein the networked PCB includes the microcontroller and the wireless transceiver to enable operating the heating pad.

21. The smart heating pad system, according to claim 18, wherein the microcontroller, the wireless transceiver, the cellular and/or satellite wireless gateway, the temperature sensor, the rechargeable battery, the 9-axis Inertial measurement unit, the gyroscope, the accelerometer, the magnetometer, and the GPS are contained in a battery holder.

22. The smart heating pad system, according to claim 19, wherein the power and communication lines extend from the battery holder.
